(19) United States
(12) Patent Application Publication
(10) Pub. No.: US 2003/0032143 A1
(43) Pub. Date: Feb. 13, 2003

(54) Collagen type I and type III compositions for use as an adhesive and sealant

(76) Inventors: Thomas B. Neff, Atherton, CA (US); Kari I. Kivirikko, Oulu (FI)

Correspondence Address: Leanne C. Price, FibroGen, Inc., 225 Gateway Blvd., South San Francisco, CA, 94080, US

(21) Appl. No.: 10/265,033

(22) Filed: Oct. 4, 2002

Related U.S. Application Data
(63) Continuation of application No. 09/621,711, filed on Jul. 24, 2000.

(51) Int. Cl. A61K 38/17; C07K 17/00; C07K 16/00; C07K 14/00; C09H 1/00; C07K 1/00; C12N 15/74; C12N 15/63; C12N 15/70; C12N 15/09; C12N 15/00; C12P 21/06; C07H 21/04; A61K 38/00

(52) U.S. Cl. 435/69.1; 530/356; 514/12; 435/320.1; 536/23.5

(57) ABSTRACT

Polymerized type I and/or III collagen based compositions for medical use as adhesives and sealants and preparation thereof are described. Prior to polymerization, the collagen monomers are prepared recombinantly whereby chemical modifications of the collagen are not needed to form such monomers. The type I and/or III collagen compositions are useful as medical adhesives for bonding soft tissues or in a sealant film for a variety of medical uses. In a further aspect of the present invention, the polymerized type I and/or III collagen composition includes agents which induce wound healing or provide for additional beneficial characteristics desired in a tissue adhesive and sealant.

This application is a continuation-in-part to U.S. Provisional Patent Application Serial No: 60/053,872 filed Jul. 28, 1997.

### 1. FIELD OF THE INVENTION

The present invention is directed to polymerized recombinant type I and/or type III collagen based compositions and combinations thereof for medical use as adhesives and sealants and the preparation of such compositions. The recombinant type I and type III collagen compositions are useful as medical adhesives for bonding soft tissues or in a sealant film for a variety of medical uses, including in wound closure devices and tendon wraps for preventing the formation of adhesion following surgical procedures. In a further aspect of the present invention, the polymerized type I and type III collagen composition includes agents which induce wound healing or provide for additional beneficial characteristics desired in a tissue adhesive and sealant.

### 2. BACKGROUND OF THE INVENTION

Mechanical, Chemical, Synthetic and Autologous Adhesion Techniques. The ability to bond biological tissues is a goal of biomedical researchers. Attempts to provide desired adhesion through mechanical bonding have proven to be neither convenient nor permanent (Buonocore, M., Adhesion in Biological Systems, R. S. Manly, ed., Academic Press, New York, 1970, Chap. 15). For example, the conventional methods of choice to close incisions in soft tissue following surgery, injury and the like have been sutures and staples. These techniques and methods, however are limited by, for example, tissue incompatibility with sutures or staples which may cause painful and difficult to treat fistulas granulomas and neuromas. Sutures and staples may also tend to cut through weak parenchymatous or poorly vascularized tissue. Sutures also leave behind a tract which can allow for leakage of fluids and organisms. The needle for any suture is larger than the thread attached to it. This causes a problem as the needle tract is larger than can be filled by the thread.

In addition, limits are imposed by the required manual dexterity and eyesight of the surgeon and the excessive amount of time that is required for the use of sutures or staples in microsurgeries. Finally, even when properly applied, the joints in the gaps between the staples or sutures may be inherently weak or may structurally weaken over time and will leak.

Several investigators have worked on laser closure of wounds (White et al., 1986; White, J. V., 1989; Oz and Bass et al., 1989; White et al., 1987). Early contributions concentrated on welding tissues using lasers of different wavelengths applied directly to wound edges. Investigating the microstructural basis of the tissue fusion thus produced, Schober and coworkers proposed that there occurred a “homogenizing change in collagen with interdigitation of altered individual fibrils” (Schober et al., 1986). These investigators, as well as others, proposed that the concentrated heating of the collagen fibrils above a threshold level allowed for their cross-linking (Goosey et al., 1980; Chacon et al., 1988; Tanzer, M. L., 1973). Unfortunately, the heat necessary to allow this reaction to occur causes collateral thermal damage. Even a slight distortion, in ocular tissue for example, may have functional consequences. Also, in the event of laser weld failure, the edges of the tissues may be damaged by the original treatment and cannot be re-exposed to laser energy (Oz, 1990).

Further work attempted to enhance heat-activated cross-inking by placing a dye in the wound. It was reported that matching the absorbance of the dye with the laser wavelength, allowed an adhesive effect to be achieved with less laser power output and collateral thermal injury (Chuck et al., 1989; Foote, C. S., 1976; Oz M. C. and Chuck et al, 1989). Coupling the dye with a protein to create a tissue “solder” was also investigated. The protein of choice has been fibrinogen, and in particular autologous fibrinogen in order to avoid problems of the transfer of viral diseases through the use of blood components from pool donors. In previous applications, fibrinogen has been obtained as a fraction of whole blood. It is not pure fibrinogen, but also contains other blood elements, such as clotting factors. Application of such a protein-dye mixture in various animal models proved to be an improvement to dye alone (Oz et al., 1990; Moazami et al., 1990). Unfortunately, human application was forestalled owing to the need to isolate the needed protein (fibrinogen) from the patient prior to the procedure to avoid the risks of infection from donor plasma. Work with albumin found it to be an unsatisfactory substitute as it did not yield welds of comparable strength.

Comparisons of protein-dye versus sutured closures have found the protein-dye group to produce less of an inflammatory response, result in greater collagen production, greater mean peak stress at rupture and better cosmesis (Wider et al., 1991). Ophthalmologic application of such a tissue solder has included the sealing of conjunctival blebs (Weisz, et al., 1989), scierostomy (Odrich et al., 1989), closure of retinectomies (Wolf et al., 1989), and thermokeratoplasty (Wapner et al., 1990) using similar mixtures.

Due to the deficiencies and limitations of these mechanical means, whether sutures, staples or more recently applied laser techniques, much attention was devoted to developing synthetic polymers, e.g., cyanoacrylates, as biomedical adhesives. These plastic materials, however, have been observed to induce inflammatory tissue reaction. Moreover, the ability of these materials to establish permanent bonding under physiological conditions has yet to be fully realized.

The known toxicity associated with synthetic adhesives has led to investigations towards the development of biologically derived adhesives as bonding materials. Among such adhesives, fibrin based glues have commanded considerable attention. (See, e.g., Epstein, G. H. et al. Ann. Otol. Rhinol. Laryngol. 95:40-45 (1986); Kram, H. B et al. Arch. Surg. 119:1309-1311 (1984), Scheele, J. et al. Surgery 95:6-12 (January 1984); and Siedentop, K. H. et al. Laryngoscooe 93:1310-1313 (1983) for general discussion of fibrin adhesives). Commercial fibrin tissue adhesives are derived from human plasma and hence pose potential health risks such as adverse immunogenic reactions and transmission of infectious agents, e.g., Hepatitis B virus. Moreover, the bond strength imparted by such adhesives are relatively weak compared to collagen adhesives (see De Toledo, A. R. et al. Assoc. for Res. in Vision and Ophthalmology, Annual Meeting Abstract, Vol. 31, 317 (1990). Accordingly, there is a need for safe, effective biologically compatible tissue adhesives for biomedical applications.

More recently, combination products have been devised for use as a tissue adhesive. For example, Staindl (Ann. Otol (1979) 88:413-418) describes the use of a combination of three separately prepared substances, human fibrinogen cryoprecipitate, thrombin in the presence of calcium ion, and Factor XIII concentrate, to obtain a glue that was applied in skin graft applications, myringoplasty, repair of dural defects, hemeostatis after tonsillectomy, and tracheoplasty. In this same time frame, Immuno-AG, Vienna, Austria, began producing and commercializing a two-component “fibrin seal” system, wherein one component contains highly concentrated human fibrinogen, Factor XIII, and other human plasma proteins, prepared from pooled blood, and the other component supplies thrombin and calcium ion. The two components are added together in the presence of a fibrinolysis inhibitor. After application, the processes of coagulation and fibrin cross-linking occur. Eventually, the seal may lyse in the process of healing of the wound or trauma which accompanies the reconstruction of the tissue. Redl, H., et at., “Biomaterials 1980,” Winter, G. D., et al., eds. (1982), John Wiley & Sons, Ltd., at page 669-675, describe the development of an applicator device for this system which mixes and applies the two components of the system simultaneously. These combination systems and their uses have been described widely: Seelich, T., J Head and Neck Pathol (1982) 3:65-69; O'Connor, A. F., et al., Otolaryngol Head Neck Surg (1982) 90:347-348; Marquet, J., J Head and Neck Pathol (1982) 3:71-72; Thorson, G. K., et al., J Surg Oncol (1983) 24:221-223. McCarthy, P. M., et al., Mayo Clin Pros (1987) 62:317-319, reported the addition of barium ion to this fibrin glue system in the treatment of a bleeding duodenal sinus in order to facilitate follow-up surveillance. See also Portmann M., J Head and Neck Pathol (1982) 3:96; Panis, R., ibid., 94-95.

Efforts have also recently focused on methods which seek to avoid the health issues raised by the use of blood plasma derived products in commercially available tissue adhesive products and systems. To this end, attempts have been made to varying degrees of success to isolate an autologous counterpart of the fibrinogen-containing component. For example, see. Feldman, M. C., et al., Arch Otolaryngol-Head and Neck Surg (1988) 114:182-185; Feldman, M. C., et al., Arch Ophthalmol (1987) 105:963-967; Feldman, M. C., et. al., M J Otolog (1988) 9:302-305; Silberstein, L. E., et al., Transfusion (1988) 28:319-321. Use of autologous fibrinogen preparations also have obvious limitations.

Collagen As A Biomaterial. Collagen, the major connective tissue protein in animals, possesses numerous characteristics not seen in synthetic polymers. Characteristics of collagen often cited include good compatibility with living tissue, promotion of cell growth, and absorption and assimilation of implantations (Shimizu, R. et al. Biomat. Med. Dev. Art. Org., 5(1): 49-66 (1977)). Various applications of this material are being tested, for example, as dialysis membranes of artificial kidney (Sterzel, K. H. et al. Ameri. Soc. Artif. Int. Organs 17:293 (1971)), artificial cornea (Rubin, A. L. et al. Nature 230:120 (1971) and U.S. Pat. No. 4,581,030), vitreous body (Dunn, M. et al. Amer. Soc. Artif. Int. Organs 17:421 (1971)), artificial skin and blood vessels (Krajicek, M. et al. J. Surg. Res. 4, 290 (1964)), as hemostatic agents (U.S. Pat. No. 4,215,200), soft contact lens (U.S. Pat. Nos. 4,264,155; 4,264,493; 4,349,470; 4,388,428; 4,452,925 and 4,650,616) and in surgery (Chvapil, M. et al. Int. Rev. Conn. Tiss. Res. 6:1-61 (1973)).

Natural collagen fibers, however, are basically insoluble in mature tissues because of covalent intermolecular cross-links that convert collagen into an infinite cross-linked network. Dispersal and solubilization of native collagen can be achieved by treatment with various proteolytic enzymes which disrupt the intermolecular bonds and removes immunogenic non-helical end regions without affecting the basic, rigid triple-helical structure which imparts the desired characteristics of collagen (see also, U.S. Pat. Nos. 3,934,852; 3,121,049; 3,131,130; 3,314,861; 3,530,037; 3,949,073; 4,233,360 and 4,488,911 for general methods for preparing purified soluble collagen).

Various methods and materials have been proposed for modifying collagen to render it more suitable as biomedical adhesives. (See, e.g., De Toledo, A. R. et al. Assoc. for Res. in Vision and Ophthalmology, Annual Meeting Abstract, Vol. 31, 317 (1990); Lloyd et al., “Covalent Bonding of Collagen and Acrylic Polymers,” American Chemical Society Symposium on Biomedical and Dental Applications of Polymers, Polymer Science and Technology, Vol. 14, Plenum Press (Gebelein and Koblitz eds.), New York, 1980, pp. 59-84; Shimizu et al., Biomat. Med. Dev. Art. Org., 5(1): 49-66 (1977); and Shimizu et al., Biomat. Med. Dev. Art. Org., 6(4): 375-391 (1978), for general discussion on collagen and synthetic polymers.). In many instances, the prior modified collagen-based adhesives suffer from various deficiencies which include (1) cross-linking/polymerization reactions that generate exothermic heat, (2) long reaction times, and (3) reactions that are inoperative in the presence of oxygen and physiological pH ranges (Lee M. L. et al. Adhesion in Biological Systems, R. S. Manly, ed., Academic Press, New York, 1970, Chap. 17). Moreover, many of the prior modified collagen-based adhesives contain toxic materials, rendering it unsuitable for biomedical use (see, for example, Buonocore, M. G. (1970) and U.S. Pat. No. 3,453,222).

Additionally, the use of collagen-based adhesives also presents immunological concerns as such adhesives have been derived from animal sources and typically bovine sources. Studies with respect to the use of such collagens as injectible devices have reported minor inflammatory responses. More recently, potential issues regarding the transmission of disorders to humans related to bovine spongiform encephalopathy (“mad cow disease”) have focused attention, especially in Europe, to limiting bovine sourced materials.

Notwithstanding these deficiencies, certain collagen-based adhesives, reportedly having appropriate adhesive strength and utility in many medical applications, particularly involving soft tissues, have been described. U.S. Pat. No. 5,219,895). These reports identify the use of type I and type II in collagen-based adhesives; wherein purified collagen types I and II are chemically modified to form monomers which are soluble at physiological conditions and then polymerized to form a composition having adhesive and sealant properties. The reports are limited to collagen-based adhesives which are composed of collagens derived from natural sources; and consequently, represent a collagen mixture. For example, type I collagen, as isolated from natural sources are typically comprised of approximately 10-20% type III and other collagens, depending upon the tissue source used, and 90-80% type I collagen.

The reports further do not refer to collagen type III, the unexpected hemostatic characteristics of type III collagen or the use of recombinant collagens so that the first chemical modification step may be avoided.

### 3. SUMMARY OF THE INVENTION

A biologically compatible, collagen type III and/or type I product with sealant and adhesive properties can be formed using soluble recombinantly derived collagen type III and/or type I monomers; wherein said monomers are polymerized to form a collagen type III and/or type I composition having adhesive and sealant properties. Preferably, the collagen is human and derived using recombinant technology. Collagen type III was selected for its unexpectedly superior hemostatic characteristics, as compared to other collagen types. Collagen type I was selected for its structural characetristics. The polymerization reaction may be initiated with an appropriate polymerization initiator such as a chemical oxidant, ultraviolet irradiation, a suitable oxidative enzyme or atmospheric oxygen.

For purposes of optimizing the sealant and adhesive properties of the recombinant collagen product by optimizing the structural stability of the product as well as the hemostatic characteristics or the product, the product is comprised preferably of a combination of pure recombinant type I and type III collagen The ratio of pure recombinant collagen type III to pure recombinant type I is about 30% and greater type III collagen to about 70% or less type I collagen. More preferably, the ratio of pure recombinant type III collagen to pure recombinant type I collagen is about 30% to about 50% type III collagen to about 70% to about 50% type I collagen. Most preferably, the ratio of pure recombinant type III collagen to pure recombinant type I collagen is about 30% to about 40% type III collagen to about 70% to about 60% type I collagen.

It is the object of this invention to provide for a pure recombinant collagen type III tissue sealant, a pure recombinant type I tissue sealant or a pure recombinant collagen type I and type III tissue sealant, free from other collagen types I, having the following characteristics and capabilities:

(i) Hemostasis. The sealant acts as a hemostatic barrier and reduces the risk of serum, lymph and liquor leakage. As collagen type III possesses inherently hemostatic properties, its use in a hemostatic device provides an improvement over known fibrin sealants. Collagen type I also possesses some hemostatic properties.

(ii) Glueing. Due to its adhesive properties, the sealant atraumatically connects tissues by forming a strong joint between them and adapts uneven wound surfaces. This glueing effect is increased by a combination of agents, as described below, and collagen type III and/or collagen type I.

(iii) Wound healing. The sealant promotes the growth of fibroblasts which in combination with efficient hemostasis and adhesion between the wound surfaces provides for an improved healing process. The use of the compositions according to the invention as an anti-adherence/wound healing composition is expected to result in a normal (regenerative) tissue rather than scar tissue, i.e. optimal wound healing. Furthermore, such compositions also reduce the inflammatory response.

Accordingly, it is an object of the present invention to provide polymerized collagen type III and/or type I compositions as a safe, effective biological adhesives with appropriate adhesive strength for biomedical applications, particularly involving soft tissues. More specifically, the present invention is directed to compositions useful in sealing punctures and incisions in internal organs, the dermis and large blood vessels. The polymerized materials may assume a number of sizes and shapes consistent with their intended biomedical applications, which include use in ophthalmology, plastic surgery, orthopedics and cardiology.

In another object of the invention, the collagen type III and/or type I composition is further comprised of agents which will confer additional desirable characteristics for a sealant or adhesive. For example, fibrin, fibrinogen, thrombin, calcium ion, Factor XIII may be included in the composition to better effect the formation of a three-dimensional network of polymerized collagen. In yet another object of the invention, the recombinant collagen type III composition incorporates a drug having wound healing capabilities. In one embodiment, the drug is connective tissue growth factor and is incorporated in the composition to effect slow-release of the drug to the wound.

### 4. DETAILED DESCRIPTION OF THE INVENTION

4.1 Definitions

As employed herein, the term “biologically compatible” refers to recombinant collagen type III and/or type I modified in accordance with the present invention (i.e., a polymerized collagen type III recombinant product) which is incorporated or implanted into or placed adjacent to the biological tissue of a subject and more particularly, does not deteriorate appreciably over time or induce an immune response or deleterious tissue reaction after such incorporation or implantation or placement.

As employed herein, the term “pure recombinant collagen type I” refers to human collagen type I manufactured by recombinant techniques which is substantially free from other collagen types. The term excludes collagen type I isolated from natural sources.

As employed herein, the term “pure recombinant collagen type III” refers to human collagen type I manufactured by recombinant techniques which is substantially free from other collagen types. The term excludes collagen type III isolated from natural sources.

4.2 Preparation of Polymerized Recombinant Collagen Type I and III

Production of Collagen Type I and III Monomers. The type of collagen useful to form the biologically compatible collagen product with adhesive and hemostatic properties of this invention is recombinant collagen type I and III. Monomeric soluble collagen type I and III is obtained by recombinant processes, including processes involving the production of collagen type III in transgenic animals. Said recombinant processes are set forth at U.S. Pat. No. 5,593,859, which is incorporated herein by reference. Preferably, collagen type I or III will be recombinantly manufactured by culturing a cell which has been transfected with at least one gene encoding the polypeptide comprising collagen type I or III and genes encoding the α and β subunits of the post-translational enzyme prolyl 4-hydroxylase and purifying the resultant collagen monomer therefrom. Preferably, the monomeric soluble collagen type I and III material exhibits a viscous consistency and varying degrees of transparency and clarity.

Polymerization Of Collagen Type I and III Monomers. The recombinant collagen type I and III solution may be subsequently subjected to polymerization or cross-linking conditions to produce the polymerized collagen composition of the present invention. Polymerization may be carried out using irradiation, e.g., UV, gamma, or fluorescent light. UV irradiation may be accomplished in the short wave length range using a standard 254 nm source or using UV laser sources. With a standard 254 nm source, 4-12 watts, polymerization occurs from 10 to 40 minutes, preferably 20 to 30 minutes, at an exposure distance of from 2.5-10 cm, preferably from 2.5 to 5 cm distance. Excess UV exposure will begin to depolymerize the collagen polymers. Polymerization using gamma irradiation can be done using from 0.5 to 2.5 Mrads. Excess Gamma exposure will also depolymerize collage polymers. Polymerization in the presence of oxygen can be done by adding an initiator to the fluid prior to exposure. Non-limiting examples of initiators include sodium persulfate, sodium thiosulfate, ferrous chloride tetrahydrate, sodium bisulfite and oxidative enzymes such as peroxidase or catechol oxidase. When initiators are employed, polymerization occurs in 30 seconds to 5 minutes, usually from 1 to 3 minutes.

The polymerizing agent is preferably UV irradiation. However, the polymerization or cross-linking of the monomeric substituents can be carried out by simply exposing the material to atmospheric oxygen, although the rate of polymerization is appreciably slower than in the case of UV irradiation or chemical agents.

Other agents may also be useful in the polymerization process. For example, to improve the cohesive strength of adhesives formed from the compositions of this invention, difunctional monomeric cross-linking agents may be added to the monomer compositions of this invention to effect polymerization. Such cross-linking agents are known in the art, for example, to U.S. Pat. No. 3,940,362 (Overhults), which is hereby incorporated by reference herein.

4.3 Collagen Type I and III Compositions

The compositions of the present invention are comprised of polymerized type I and III collagen wherein said composition is manufactured by a process comprising the steps: (1) production of collagen type I and III monomers by the recombinant methods described above; and (2) polymerization of such monomers.

For purposes of optimizing the sealant and adhesive properties of the recombinant collagen product by optimizing the structural stability of the product as well as the hemostatic characteristics or the product, the product is comprised preferably of a combination of pure recombinant type I and type III collagen The ratio of pure recombinant collagen type III to pure recombinant type I is about 30% and greater type III collagen to about 70% or less type I collagen. More preferably, the ratio of pure recombinant type III collagen to pure recombinant type I collagen is about 30% to about 50% type III collagen to about 70% to about 50% type I collagen. Most preferably, the ratio of pure recombinant type III collagen to pure recombinant type I collagen is about 30% to about 40% type III collagen to about 70% to about 60% type I collagen.

The compositions of the present invention may be further comprised of other agents which are useful to glueing or sealing tissues. For example, in addition to recombinant collagen type I and/or type III protein, the composition will preferably comprise Factor XIII and/or fibrin/fibrinogen/fibronectin and/or plasminogen. Advantageously, the composition will also include clotting enzyme, i.e. thrombin, especially in combination with bivalent calcium, such as calcium chloride. The concentration of calcium chloride will then vary, e.g. between 40 mM to 0.2M depending on the specific purpose of the tissue adhesive composition, high concentrations of calcium chloride inhibiting fibroblast growth and therefore being preferred for anti-adherence applications (along with absence of fibronectin which stimulates the growth of fibroblasts). It may further be valuable to include a fibrinolysis inhibitor, such as a plasmin inhibitor, e.g. aprotinin, aprilotinin, alpha-2-antiplasmin, alpha-2-macroglobulin, alpha-1-antitrypsin, epsilon-aminocaproic add or tranexamic acid, or a plasmin activator inhibitor, e.g. PAI-1 or PAI-2.

While the proportions of the previously known ingredients in the tissue adhesive compositions of the invention may be selected with guidance of prior art compositions, the necessary amount of the viscosity enhancing polymer: can readily be determined by a person skilled in the art depending on the particular polymer and the intended use form. Thus, if the concentration and/or molecular weight of the viscosity enhancing polymer is too low, the viscosity increase will be insufficient, and a too high concentration and/or molecular weight will inhibit the fibrin polymerization and the adhesion to the tissue.

By increasing the thrombin concentration, the polymerization of composition of the present invention may be speeded up with a consequential influence on the time until the glue sets. At low thrombin concentrations, for example, the fibrin of the composition will remain more or less fluid for several minutes after application. A further beneficial effect of increasing the viscosity with a viscosity enhancing polymer in accordance with the invention is therefore the possibility to use lower concentrations of thrombin, which is required in situations where the parts to be sealed require subsequent adaptation even on non-horizontal surfaces.

Likewise, the compositions of the present invention may, rather than including a combination of the agents described herein, be a fusion protein wherein the collagen type I and/or type III and, for example, fibrin, are combined to form one molecule. Such fusion proteins may be manufactured according to the recombinant techniques described herein.

In a further embodiment of the invention, the composition of the present invention includes agents useful in wound healing, either by inducing or promoting the formation of tissue, or alternatively, limiting the formation of fibrotic adhesions. Such agents include antibiotics, or growth factors, such as connective tissue growth factor, which is described at, for example, U.S. Pat. No. 5,408,040 and 5,585,270, said references are incorporated herein by reference.

4.4 Fields Of Use

The polymerized collagen type III and/or type I product may be useful to produce mechanical sealants and adhesive systems.

Tissue Adhesive Systems. Fields of application include among others: ear, nose and throat surgery, general surgery, dentistry, neurosurgery, plastic surgery, thorax and vascular surgery, abdominal surgery, orthopaedics, accident surgery, gynaecology, urology, and opthalmology. The collagen sealants of the present invention have also been used for local application of drugs, such as antibiotics, growth factors and cytostatics.

Sealant Films. In one aspect of the invention, the polymerized collagen products can be made in the form of a sealant film. A collagen based film will be flexible and elastic with the consistency and feel of plastic film, and yet the film should exhibit high biological compatibility. Uses of sealant films include: Prevention of adhesion formation following tendon surgery (i.e., use as a wrap around tendons), use as a synthetic tympanic membrane, substitute facial tissue and wound dressing component. Additional examples of potential usage of sealant films include: treatment of corneal abrasions, wound closure, coating of catheters and instruments, use as a material to prevent adhesion formation in tissues than tendons (e.g., peritoneal cavity).

Further embodiments of the present invention include sealant and adhesive formulations which can be used as systems specific for delivery of numerous drugs and pharmaceutical compositions, including growth factors, antibiotics, and other biologically beneficial compounds. Such materials can be added to the collagen adhesive or sealant to promote cell migration, cell adhesion, and wound healing.

Angioplasty and Angiography. Angiography is a diagnostic procedure whereby dye is injected into an artery, preferably the femoral artery, to detect the presence or absence of coronary disease. Angioplasty, also known as PCTA, is a therapeutic procedure which involves the inflation of a balloon in an artery, such as the coronary artery, for the purpose of relieving arterial blockages. After puncturing the femoral artery, a balloon-catheter is introduced through the femoral artery and navigated through to the coronary artery blocked by atherosclerosis (plaque). Once in position, the balloon is inflated and deflated several times in an effort to open the artery by pushing the fatty material against the vessel walls, allowing for blood to circulate to the affected regions of the heart muscle. Various types of balloon catheters are commonly used in angioplasty and angiography including over-the-wire catheters which ride over an independent guidewire to the site of the disease; 2) fixed-wire catheters, which combine a balloon catheter with a guidewire into one device; 3) rapid-exchange or single-operator exchange catheters, which are over-the-wire catheters that can be exchanged more conveniently than standard over-the-wire catheters; and 4) perfusion catheters, which allow blood flow during the procedure. A rotational tip catheter removes plaque buildup on arterial walls. These devices utilize a technique called differential cutting. Calcified material is rendered into microscopic particles without damaging the artery due to the elastic nature of the arterial walls.

Angioplasty is a more invasive and complicated procedure than angiography, since it requires the insertion of a larger sheath than that used in angiography. The sheath is used as a vehicle for introducing the catheter into the artery. Additionally, angioplasty also requires the use of blood thinners, such as heparin, to prevent clotting during and after the surgical procedure. The anti-clotting agent prevents the body's natural sealing/clotting mechanism and, thus, sealing punctures requires a significant length of time.

According to the present invention, after withdrawing the catheter and other invasive devices from the artery, an adhesive applicator may optionally be inserted into the sheath and is placed into a position near to or contacting the puncture in the artery. During the procedure, manual or mechanical pressure is applied to the artery to reduce the flow of blood at the puncture site. If possible, excess blood/fluid is removed from the puncture site. Subsequently, recombinant collagen type III and/or type I monomer of the present invention may be applied to the puncture on the external surface of the artery and/or within the puncture track. The monomer then is polymerized and/or cross-linked by the techniques described herein, for example, UV irradiation, such that polymerization takes place within 0 to 300 seconds, preferably 0 to 120 seconds, more preferably 0 to 30 seconds, and even more preferably 3 to 10 seconds. By applying the collagen monomer composition on the outside of the artery, the incidence of embolism (blockage of the artery or circulatory system) is virtually eliminated. Alternatively, a polymerized collagen type III and/or type I may be used and the polymerization step may be avoided. Because of the bonding strength of the adhesive of the present invention, only small amounts of the adhesive are required to seal a punctured artery. Moreover, because the surgical adhesive according to the present invention can polymerize almost immediately, the adhesive can polymerize on the surface and/or along the puncture track of the artery without penetrating the interior of the artery. Accordingly, large pieces or particles of material will not enter the circulatory system, thereby substantially reducing risk of embolism. Due to the fast and strong bonding of preferred adhesives of the invention, the patient will need to be immobilized for only a minimal period of time.

4.5 Administration

Formulations. The tissue treatment composition of the present invention may be presented in the same type of preparations as the prior art fibrin sealants. The components may be provided in deep frozen solution form or as lyophilized powders, to be diluted prior to use with appropriate aqueous solutions, e.g. containing aprotinin and calcium ions, respectively.

With respect to compositions of the present invention comprising pharmaceutical agents, such as an antibiotic, a growth factor, etc, by incorporating said agent into the tissue adhesive so as to be enclosed in the collagen network formed upon application of the tissue adhesive. It will thereby be ensured that the drug is kept at the site of application while being controllably released from the composition, e.g. when used as ocular drops, a wound healing preparation, etc. As also mentioned above the pharmaceutically active substance to be released from the present tissue adhesive composition may be the viscosity enhancing polymer in itself or a substance coupled thereto. A specific example of such a viscosity enhancing polymer fulfilling the viscosity enhancing requirement as well as having therapeutical and pharmaceutical utility, and for which it may be desired to sustain the bioavailability, is hyaluronic acid and salts and derivatives thereof which are easily soluble in water and, as mentioned previously, have an extremely short biological half-life. The tissue treatment composition of this invention thus constitutes an advantageous slow-release preparation for proteoglycans such as hyaluronic acid and its salts and derivatives, and considerably increases the bioavailability thereof.

Notably, the compositions of the present invention are not restricted to the adhesive properties, but non-adhesive compositions are also included, especially when the compositions primarily are intended for wound healing. The latter compositions may in particular include non-adhesive proteins such as albumin and/or growth factors. Substantially non-adhesive compositions may also be obtained when the polymer part of the composition inhibits the adhesive properties of the protein part. It should in this context be emphasized that the invention comprises both adhesive and substantially non-adhesive compositions, although it has for simplicity reasons often has been referred to as an “adhesive” in this specification.

Application Of Compositions. The compositions of the present invention may be applied using a variety of dispensing devices. For example, the surgical adhesive may be applied using the devices set forth in U.S. Pat. Nos. 4,900,303 (Lemelson) and 5,372,585 (Tiesenbrun) while monitoring the application process through an optical viewing system. The composition of the present invention may also be applied by the devices set forth in U.S. Pat. No. 5,129,882 (Weldon et al.). The subject matter of these patents is incorporated herein by reference.

The composition according to the present invention may also be applied in conjunction with other sealing means. For example, the adhesive may be applied to puncture sites which have been closed using surgical suture or tape, such as in the sealing of a puncture or incision in internal organs, e.g., liver, gallbladder, intestines, stomach, kidney, heart, urinary bladder, ureter, lung, esophagus and the like. The adhesive in this instance will provide a complete seal, thereby reducing the risk of body fluid leakage from the organ or vessel, e.g., leakage from liver puncture sites. The surgical adhesive of the present invention may additionally be used in conjunction with other sealing means, such as plugs, and the like. Such techniques are set forth in U.S. Pat. Nos. 4,852,568 (Kensey), 4,890,612 (Kensey), 5,053,046 (Janese), 5,061,274 (Kensey), 5,108,421 (Fowler), 4,832,688 (Sagae et al), 5,192,300 (Fowler), 5,222,974 (Kensey et al.), 5,275,616 (Fowler), 5,282,827 (Kensey et al.), 5,292,332 (Lee), 5,324,306 (Makower et al.), 5,370,660 (Weinstein et al.), and 5,021,059 (Kensey et al.). The subject matter of these patents is incorporated herein by reference.

Notably, the compositions of this invention can be used to join together two surfaces by applying the particular composition to at least one of said surfaces. Depending on the particular requirements of the user, the adhesive compositions of this invention can be applied by known means such as with a glass stirring rod, sterile brush or medicine dropper; however, in many situations a pressurized aerosol dispensing package is preferred in which the adhesive composition is in solution with a compatible anhydrous propellant. Aerosol application of the monomers is particularly advantageous for use in hemostasis.

What is claimed is:

1. A tissue adhesive or sealant composition comprising a polymerized collagen type III wherein said adhesive or sealant composition is produced by recombinantly manufacturing pure collagen type III monomers in a cell and polymerizing said monomers with an agent.

2. The composition of claim 1 wherein the composition is biologically compatible.

3. The composition of claim 1 wherein the recombinant manufacture of a collagen type III monomer comprises the following steps:
(a) culturing a cell which has been transfected with at least one gene encoding a polypeptide comprising collagen type III and at least one gene encoding a polypeptide selected from the group the α or β subunit of prolyl 4-hydroxylase; and
(b) purifying said collagen type III.

4. The composition of claim 1 wherein the said monomers are polymerized using irradiation.

5. The composition of claim 4, wherein said irradiation is UV irradiation.

6. The composition of claim 1 wherein the composition is further comprised of one or more agents selected from the group fibrin, fibrinogen, thrombin, Factor XIII or connective tissue growth factor.

7. A process for making a tissue sealant or adhesive comprising the steps:
(a) manufacturing collagen type III monomers by recombinant means; and
(b) polymerizing said collagen type III monomers.

8. A tissue adhesive or sealant composition comprising a polymerized collagen type I wherein said adhesive or sealant composition is produced by recombinantly manufacturing pure collagen type I monomers in a cell and polymerizing said monomers with an agent.

9. The composition of claim 8 wherein the composition is biologically compatible

10. The composition of claim 8 wherein the recombinant manufacture of a collagen type I monomer comprises the following steps:
(a) culturing a cell which has been transfected with at least one gene encoding a polypeptide comprising collagen type I and at least one gene encoding a polypeptide selected from the group the α or β subunit of prolyl 4-hydroxylase; and
(b) purifying said collagen type I.

11. The composition of claim 8 wherein the said monomers are polymerized using irradiation.

12. The composition of claim 11, wherein said irradiation is UW irradiation.

13. The composition of claim 8 wherein the composition is further comprised of one or more agents selected from the group fibrin, fibrinogen, thrombin, Factor XIII or connective tissue growth factor.

14. A process for making a tissue sealant or adhesive comprising the steps:
(a) manufacturing collagen type I monomers by recombinant means; and
(b) polymerizing said collagen type I monomers.

15. A tissue adhesive or sealant composition comprising a polymerized pure collagen type III and a polymerized pure collagen type I.

16. The composition of claim 15 wherein the composition is biologically compatible.

17. The composition of claim 15 wherein the ratio of pure recombinant collagen type III to pure recombinant collagen type I is about 30% or greater collagen type III to about 70% or less collagen type I.

18. The composition of claim 15 wherein the said monomers are polymerized using irradiation.

19. The composition of claim 15, wherein said irradiation is UV irradiation.

20. The composition of claim 15 wherein the composition is further comprised of one or more agents selected from the group fibrin, fibrinogen, thrombin, Factor XIII or connective tissue growth factor.
